(19) United States
(12) Patent Application Publication
(10) Pub. No.: US 2015/0157880 A1
(43) Pub. Date: Jun. 11, 2015

(54) CLOUD-BASED ADAPTIVE QUALITY ASSURANCE FACILITIES

(71) Applicant: IMAGE OWL INC., Salem, NY (US)

(72) Inventors: Michael Geoffrey Dalbow, Eagle, CO (US); Smari Kristinsson, Hafnarfjordur (IS); Asbjörn H. Kristbjornsson, Reykjavik (IS); Hildur Olafsdottir, Seltjarnarnes (IS); Jesper Fredriksson, Skondal (SE)

(73) Assignee: IMAGE OWL INC., Salem, NY (US)

(21) Appl. No.: 14/560,489

(22) Filed: Dec. 4, 2014

Related U.S. Application Data
(60) Provisional application No. 61/912,248, filed on Dec. 5, 2013.

(51) Int. Cl. A61N 5/10

(52) U.S. Cl. CPC A61N 5/1075

(57) ABSTRACT

Presented herein are facilities to assist in quality assurance (QA) testing of systems and equipment. Test data including results related to radiologic or diagnostic imaging performance of radiation therapy or diagnostic imaging equipment is obtained. Analysis of the test data is performed against maintained quality assurance specifications to obtain quality assurance results for the equipment. The obtained quality assurance results are presented to a user. Users may view trends and comparison in QA testing for the equipment and other equipment. Alerts about QA results may be provided and comments on QA results may be maintained. Collaboration between users to share information, test data, advice, and so on is facilitated.

### CROSS REFERENCE TO RELATED APPLICATION

This application claims priority under 35 U.S.C. §119(e) to U.S. Provisional Patent Application No. 61/912,248 filed Dec. 5, 2013 entitled, “CLOUD-BASED ADAPTIVE QUALITY ASSURANCE FACILITIES”, the content of which is hereby incorporated herein by reference in its entirety.

### BACKGROUND

Radiation therapy and diagnostic imaging equipment undergoes periodic testing to ensure compliance with various standards, optimal function to meet clinical requirements, and, ultimately, to protect operators and patients from over-exposure or under-exposure to potentially harmful magnetic or ionizing radiation fields. Quality assurance (QA) test data acquired for the periodic tests is typically stored at the facility housing the equipment. A site medical physicist is responsible for recognizing suboptimal equipment function, performing device recalibration, and signing-off on any issues that arise. Thus, the acquisition of QA test data, review thereof, and response thereto is localized to the responsible site and its personnel.

### BRIEF SUMMARY

Shortcomings of the prior art are overcome and additional advantages are provided through the provision of a method that includes obtaining test data, the test data comprising results related to radiologic or diagnostic imaging performance of radiation therapy or diagnostic imaging equipment; performing analysis of the test data against maintained quality assurance (QA) specifications to obtain QA results for the equipment; and presenting the obtained QA results to a user.

Further, a computer system is provided that includes a memory and a processor in communication with the memory, and the computer system is configured to perform: obtaining test data, the test data comprising results related to radiologic or diagnostic imaging performance of radiation therapy or diagnostic imaging equipment; performing analysis of the test data against maintained quality assurance (QA) specifications to obtain QA results for the equipment; and presenting the obtained QA results to a user.

Yet further, a computer program product is provided. The computer program product includes a non-transitory computer-readable storage medium having program instructions for execution by a processor to perform: obtaining test data, the test data comprising results related to radiologic or diagnostic imaging performance of radiation therapy or diagnostic imaging equipment; performing analysis of the test data against maintained quality assurance (QA) specifications to obtain QA results for the equipment; and presenting the obtained QA results to a user.

The presenting of the obtained QA results can include, as examples, providing an alert to the user, the alert indicating one or more QA results, of the obtained QA results, that are out-of-specification; or providing a trend report to the user, the trend report providing a comparison of the obtained QA results to prior-obtained QA results.

The presenting may present the alert to the user, where the one or more QA results indicated by the alert identify a particular data point or test data value corresponding to that data point that is out of specification.

The method can further include aggregating the obtained QA results with additional QA results for other radiation therapy or diagnostic imaging equipment. The prior-obtained QA results can include the additional QA results for the other radiation therapy or diagnostic imaging equipment, where the presenting presents the trend report to the user, and where the trend report provides a comparison of the obtained QA results to the additional QA results. The obtained QA results may be for one system and the additional QA results may be for one or more other systems, the one system and the one or more other systems being of a same type of equipment, where the trend report indicates how QA results for the one system are trending over time, and the trend report indicates how QA results for the one system compare to QA results for the one or more other systems.

The method can further include, in response to the presenting, receiving one or more comments from the user. The one or more comments may be associated with a QA result, of the obtained QA results, or with an individual test data point, where the one or more comments support, explain, resolve, comment-on, or sign-off on the QA result or individual test data point. The one or more comments may include an indication of a repair, reconfiguration, or retest performed on the equipment based on the presenting. The one or more comments may include an electronic signing-off by the user for a compliance issue indicated by QA results.

The presenting can present a message or description of a compliance issue indicated by the QA results, where the one or more include comprise a comment directed to that compliance issue.

The analysis may includes statistical process control (SPC) analysis, the SPC analysis facilitating detection of slow drift in machine performance, where the QA results includes an indication of the slow drift or predictive advice directed to further functioning of the equipment.

The maintained QA specifications may include a protocol for QA testing of the equipment, where the method further includes determining whether the obtained test data for the equipment satisfies the protocol; and providing an indication to the user about any deviation from the protocol.

The method may further include maintaining a Wiki with information about aspects of a facility in which the equipment is maintained, specification about the equipment components, information about test data points of QA testing to be performed for the equipment, or information explaining results of testing performed for the equipment.

Additional features and advantages are realized through the concepts and aspects described herein. Other embodiments and aspects of the invention are described in detail herein and are considered a part of the claimed invention.

### BRIEF DESCRIPTION OF THE DRAWINGS

One or more aspects of the present invention are particularly pointed out and distinctly claimed as examples in the claims at the conclusion of the specification. The foregoing and other objects, features, and advantages of the invention are apparent from the following detailed description taken in conjunction with the accompanying drawings in which:

FIG. 1 depicts an example interface for uploading file(s) to a cloud facility, in accordance with aspects described herein;

FIG. 2 depicts an example interface for presenting results to a user, in accordance with aspects described herein;

FIG. 3 depicts an example interface displaying machine status for machines associated with an account, in accordance with aspects described herein;

FIG. 4 depicts one example of a QA test report, in accordance with aspects described herein;

FIG. 5 presents alternative depictions of QA test data, in accordance with aspects described herein;

FIG. 6 depicts an example interface displaying a trend report, in accordance with aspects described herein;

FIG. 7 depicts an example device comparison interface in accordance with aspects described herein;

FIG. 8 depicts an example interface of a mobile application, in accordance with aspects described herein;

FIG. 9 depicts an overview of an example cloud facility in accordance with aspects described herein;

FIG. 10 depicts one example of a computer system to incorporate and use aspects described herein; and

FIG. 11 depicts one embodiment of a computer program product incorporating aspects described herein.

### DETAILED DESCRIPTION

Presented herein are facilities to assist in quality assurance (QA) testing of systems and equipment, for instance equipment used in radiology and radiation therapy treatment. Such systems and equipment are collectively referred to herein as RTT AND IMAGING systems. RTT AND IMAGING systems include radiation therapy equipment and/or diagnostic imaging systems equipment based on various technologies. Example systems/technology include, but are not limited to:
computed tomography (x-ray, CAT, single-photo emission, cone-beam CT (CBCT), megavoltage computer tomography (MVCT), etc.);
magnetic resonance (magnetic resonance imaging (MRI) including functional MRI, magnetic resonance neurography (MRN), cardiac magnetic resonance (CMR), magnetic resonance angiography (MRA), magnetic resonance cholangiopancreatography (MRCP), endorectal coil magnetic resonance imaging, delayed gadolinium enhanced magnetic resonance imaging of cartilage (dGEMRIC), magnetic resonance elastography (MRE), interventional magnetic resonance imaging (IMRI), magnetic resonance spectroscopic imaging, etc.);
positron emission tomography (PET);
fluoroscopy;
digital X-ray; ultrasound;
planar kV, MV and electronic portal imaging devices;
non-ionizing radiation imaging systems; and
therapeutic systems based on photon/electron (external beam and brachy), proton, hyperthermia, and cryotherapy technology.

Aspects described herein also apply to other types of systems, such as robotic surgery systems, and testing systems that are not radiographic in nature, such as safety/functionality testing systems.

Testing of such RTT AND IMAGING systems involves scanning/exposing a QA test device, sometimes referred to as a “phantom”, using the RTT AND IMAGING system, and collecting test data using an associated data processing/computer system. Typically, these RTT AND IMAGING systems have associated therewith one or more computer systems to drive control of the equipment and collection of data generated therefrom. The test data is formatted in any of a variety of standard or vendor-proprietary formats. An example standard format is the Digital Imaging and Communications in Medicine (DICOM) format.

In accordance with aspects described herein, test data is uploaded and stored to a cloud facility where it can be analyzed for, for instance, QA and quality control purposes. Statistical methods, such as statistical process control, may be applied in the monitoring and control of the QA process for this equipment. Test results may be presented in various forms, such as standardized or user-define templates. Statistical abnormalities and results that are out-of-specification may be highlighted and identified for the users. Alerts (GUI-based, email, text, etc) and trends may be provided to users for their review, comment, and/or resolution, as appropriate, and reports may be automatically or manually created based on the received test data. Example types of reports include those for regulatory bodies or vendors, though other types are possible.

The test data and other information, for instance images acquired using the equipment, are stored and backed up securely in a cloud-based adaptive quality assurance facility (referred to herein as a “cloud facility”). Storage of this data in the cloud facility provides safe, secure, convenient, and universal access to the data from a variety of network-connected devices. Access may be provided through web browser(s) and mobile applications (“apps”), as examples, running on any variety of computer systems having a network connection to the cloud facility. Example computer systems include desktops, laptops, and mobile devices such as those running the iOS operating system (iPhone or iPad devices, for example) or Android operating system (Nexus devices, for example) (iOS, iPhone, and iPad are trademarks of Apple Inc, Cupertino, Calif., and Android and Nexus are trademarks of Google, Inc., Mountain View, Calif.). QA data and information from many different sites having many different RTT AND IMAGING systems can be aggregated to the cloud facility. Storage in the cloud facility, as opposed to the localized storage of this data at only the individual sites, provides access by multiple users from multiple different sites (internal/external), as well as standardization, and opportunities for teaching and support based on the dataset. For instance, a Wiki teaching/information tool may be integrated into the cloud facility to provide information about the various aspects of the facility, the devices being tested, the results of the tests, etc.

As noted above, test data files can be written in any of a variety of formats. DICOM is an international standard format in the industry for storing image data. DICOM-RT (radiation therapy) is an example of a proprietary format based on the DICOM standard. Some equipment manufacturers, such as Sun Nuclear Corporation, IBA Dosimetry GmbH, Standard Imaging, Inc., and Fluke Corporation, use their own proprietary format(s) for storing data acquired from the RTT AND IMAGING systems, including test data and images.

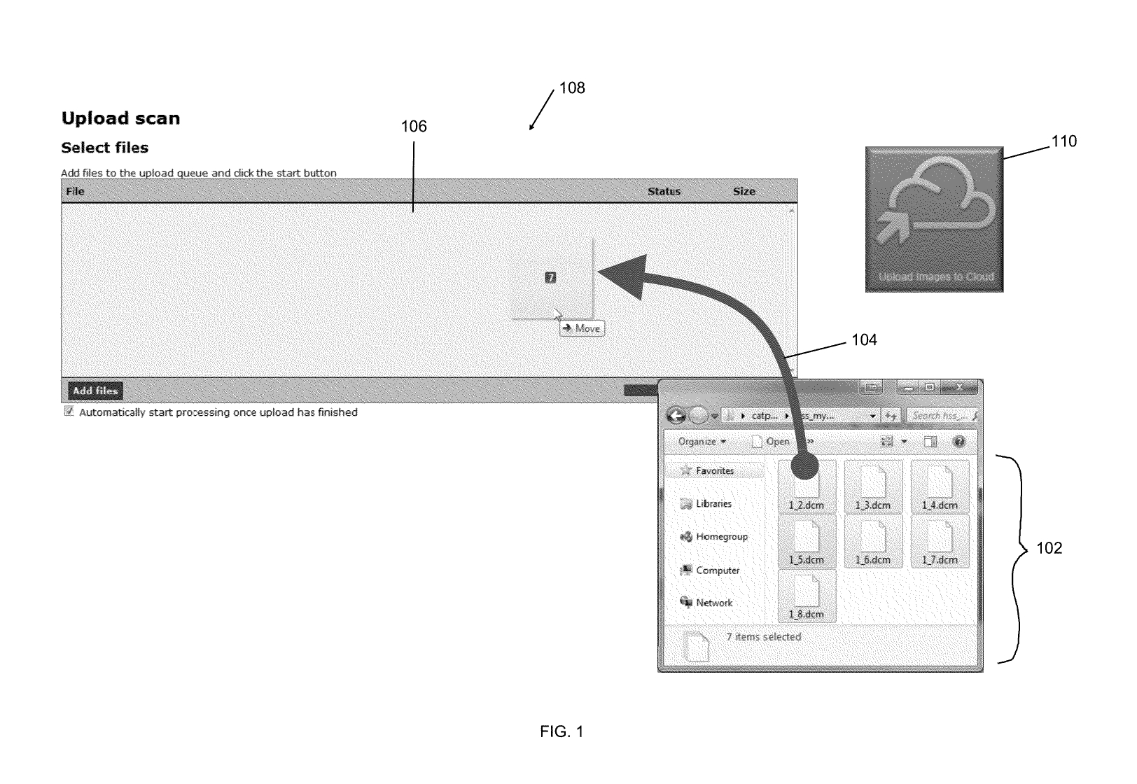

The test data files and acquired images are uploaded to the cloud facility across one or more network connections. FIG. 1 depicts an example interface for uploading file(s) to the cloud facility, in accordance with aspects described herein. RTT AND IMAGING systems typically have associated therewith a computer that stores the data in a particular file format. In FIG. 1, the test data files 102 are in the .dcm (DICOM) format. A user can navigate to the test data files stored locally, and drag and drop 104 the files into an upload queue 106 of an upload interface 108 provided in, for instance, a webpage hosted by, or in communication with, the cloud facility. Once the files are dropped into the upload queue 106, they may automatically (or manually by clicking an ‘Upload images to cloud’ button 110) upload to the cloud facility. Typically when images and/or test data files are created by an RTT AND IMAGING system at a site (such as a hospital or other health care provider facility), they are stored to a Picture Archive Communication System (PACS) or similar system. From there, in accordance with aspects described herein, this data can be uploaded to the cloud facility directly from the PACS. In some cases, the test files may be automatically uploaded, absent manual participation by a user. For instance, a client-side listener installed on computer(s) having access to the locally stored data, such as a computer system of a PACS, can recognize when new data is stored, and automatically upload the new data to the cloud facility.

Some RTT AND IMAGING or PACS systems may be configured to upload data to existing cloud-storage facilities such as DropBox or Google Drive. In these cases, as another option for upload, aspects of the cloud facility can pull test data directly from these existing cloud-storage facilities by way of one or more APIs providing access to the data.

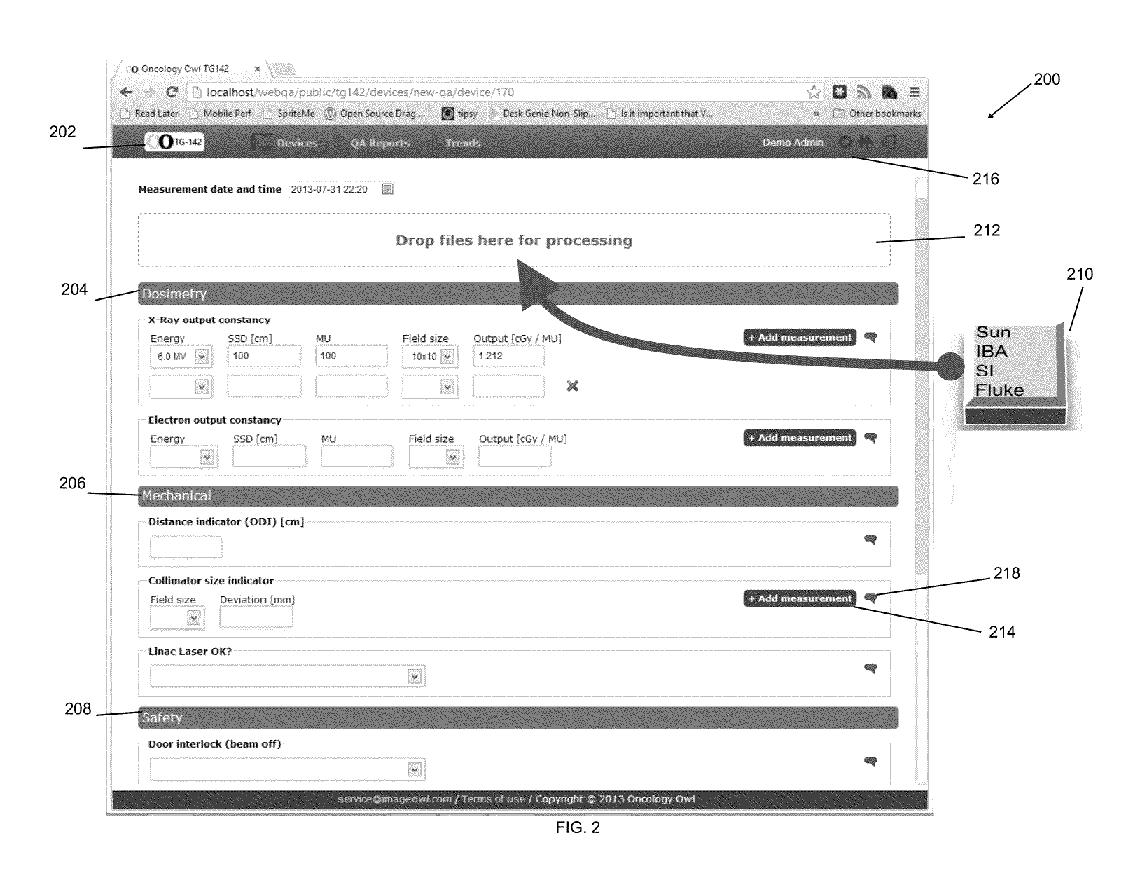

After test data files have been pushed or pulled to the cloud facility, the test data can be parsed and analyzed, and the results (QA results) can be presented to a user in various ways. FIG. 2 depicts an example interface for presenting results to a user, in accordance with aspects described herein. In some examples, the interface 200 of FIG. 2 is a web-interface presented in a web browser for the user.

Referring to FIG. 2, within interface 200 is a TG-142 indication 202 in the upper left hand corner referring to recommendations derived from a report of Task Group 142 of the American Association of Physicists in Medicine. TG 142 outlines a protocol that provides for daily, weekly, monthly and yearly tests of medical accelerators. To satisfy requirements, such as those of the TG 142 protocol, templates can be configured with the cloud facility and the templates can be populated with test data for display to a user. FIG. 2 depicts an interface showing the TG 142 daily test template having multiple different test points (Dosimetry 204, Mechanical 206, Safety 208, X-ray constancy (not pictured), etc) and subpoints within those test points. Interface 200 can initially include a blank template with no values for test points and subpoints (represented as fields in interface 200). The test points and subpoints may then be automatically populated with data based on a user dragging and dropping a test data file 210 (in any recognizable format such as Sun, IBA, SI, Fluke, etc) to the “Drop file here for processing” area 212 of interface 200. Thus, an upload interface (212) may be included within a template page, such as this TG 142 template page of FIG. 2. When the test data file is dropped into the appropriate location, the cloud facility can read off the test data file and automatically populate values for the various test points/subpoints of the template.

Continuing with the description of FIG. 2, “Add measurement” buttons (e.g. 214) are provided for a user to include additional measurements in this template. For instance, the user could click an “Add measurement” button under the X-ray output constancy test point to specify an additional X-ray output constancy (such as 12.0 MV) and manually enter the associated values. Additionally or alternatively, if data is to be provided from one or more other test date files, an “Add measurement” button may be selected and present an additional upload interface for a user to upload/extract data from a separate test data file to populate the measurement being added.

A ‘gear’ button 216 toward the top right corner of the web interface can be selected to launch a Wiki, which can house information and comments about the particular measurements, expected values, etc, as well as comments that have been entered by users. ‘Information bubble’ buttons (e.g. button 218 adjacent to “Add measurement” button 214) can be provided for the measurement(s) and, when selected, present information from the Wiki regarding/explaining the measurement(s) or test(s), and provide the ability for a user to add a comment about the measurement value(s). The user may wish to enter a comment explaining why a particular measurement is out of an expected range. If the data does not meet specification, an indicator, such as pop-up or color shading associated with the particular measurement, can be presented to the user informing that something is wrong. The user can click to view a message, description, etc. indicating what is wrong, and the user can post a comment directed to that issue. The comment can be associated with the particular account (under which this test data is uploaded), and the particular machine, test date, etc. that is the subject of this particular daily test. Thus, facilities provided herein can track and record not only the data itself but the comments (entered by users) that support, explain, resolve, comment-on, sign-off on, etc. the data.

Also in accordance with aspects described herein, a user may receive indications and alerts through a mobile application that provide the status of QA testing with respect to devices (RTT and IMAGING systems) associated with the user's account. An alert can identify a particular measurement and/or corresponding value that is out of specification and the user can respond with a comment using the mobile application. Further details are provided below.

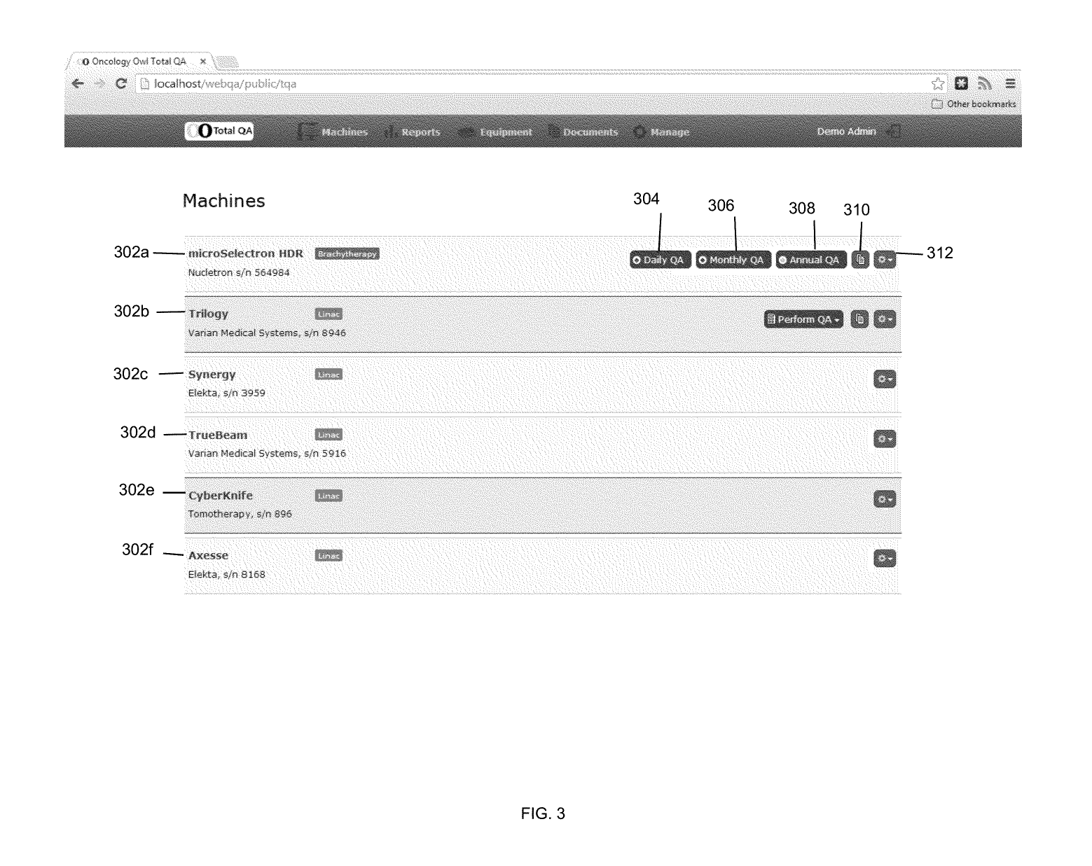

FIG. 3 depicts an example interface displaying machine status for machines associated with an account, in accordance with aspects described herein. A user can associate machine(s) with the user's account by indicating machine manufacturer and model, and providing any additional information necessary for identifying that machine to the cloud facility. In one example, this is provided by way of a web-based interface. The selection can be made from a list of machines that the cloud facility has been configured to recognize. In this regard, the cloud facility is preconfigured with specifications for various machines. In one example, the specifications are vendor-provided specifications that are loaded into the system by an administrator. Additionally, functionality can be provided to enable individual users to set up their own specifications so that they can choose any one or more specifications against which test values for a given machine are to be judged. This is useful in cases where a governing body has put into effect a different set of specifications than those put forth by the machine manufacturer.

For each device, next to the device name (e.g. 302a-302f) is an indication of the type of device (e.g., Brachytherapy, Linear accelerator (“Linac”) in this example). The interface also indicates various QA that is due for each machine. The presence of the QA buttons (“Daily QA” button 304, “Monthly QA” button 306, and “Annual QA” button 308) in association with the microSelectron HDR machine 302a indicates that these QA tests are due for the microSelectron HDR. Selection of a QA button will initiate performance of that QA for that machine. With regard to the Trilogy system 302b, only the QA under the “Perform QA” category is due for that system. “Perform QA” is used to indicate that some QA has been performed but that additional QA tests must be completed. No QA is presently needed for the other machines (Synergy 302c, TrueBeam 302d, CyberKnife 302e, Axesse 3020.

If there is a problem with a piece of equipment, for instance a QA test point is out of specification, a responsible party may be required to investigate, correct, and/or sign-off on the issue. A button (e.g. button 310 between the “Annual QA” button 308 and ‘gear’ button 312 associated with the microSelectron HDR machine) can be included that indicates that there are outstanding notes/comments for the particular device. Selecting this button will display these notes/comments. When no notes/comments are outstanding, such as after the responsible party has signed-off on the note/comment, then the button indicating outstanding issue(s) may be removed.

A gear button (e.g. 312) associated with a device provides access to information, configuration, and QA testing for the given machine. Selecting the gear button would navigate to an interface for managing the device, allowing the user to access properties of the machine and QA testing/results that has been performed or that is due for the machine.

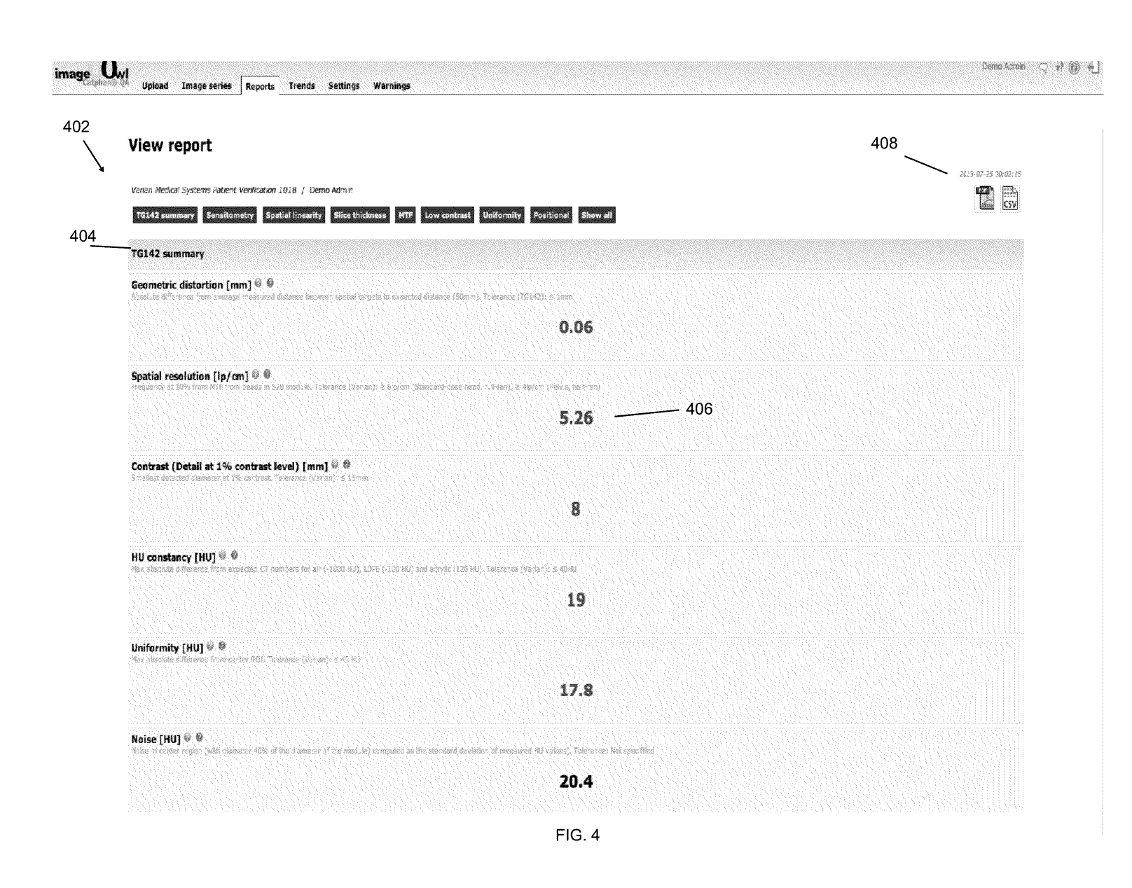

Highlighting over the entry for a machine (e.g. Trilogy, CyberKnife in this example) indicates that something was wrong as evidenced by the testing of those devices. A user can select the highlighted entry for that machine and be presented with a report of test data values for that machine, providing further detail of the testing, and indicating specific test value(s) that did not meet the specification. FIG. 4 depicts an example of a QA test report, in accordance with aspects described herein.

Along the top 402 of the interface are the tests that are setup to be completed on the device. The view of FIG. 4 depicts the TG 142 summary 404 report. In this example, the Spatial Resolution test value (406) of 5.26 lp/cm measured in this testing did not meet specification, and therefore may be highlighted in some manner, for instance altered in appearance as compared to other values (e.g. shown in different color (red), font, size, animation, highlighting, etc) to indicate that the spatial resolution test did not meet specification. Other values that did meet specification may be highlighted in a different manner (e.g. shown in green), and values for which no specification has been provided (i.e. the cloud facility has not been not configured with criteria on which to judge whether that value passed or failed) may be highlighted or presented in yet a different manner (the color black, for instance).

In the top right corner of the interface are icons 408 for selection to download the report. The reports can be downloaded in PDF or CVS format (in this example), though reports can be formatted and presented in any desired file type. Export of the reports for downloading and printing may be useful for auditing or regulatory compliance purposes, as examples.

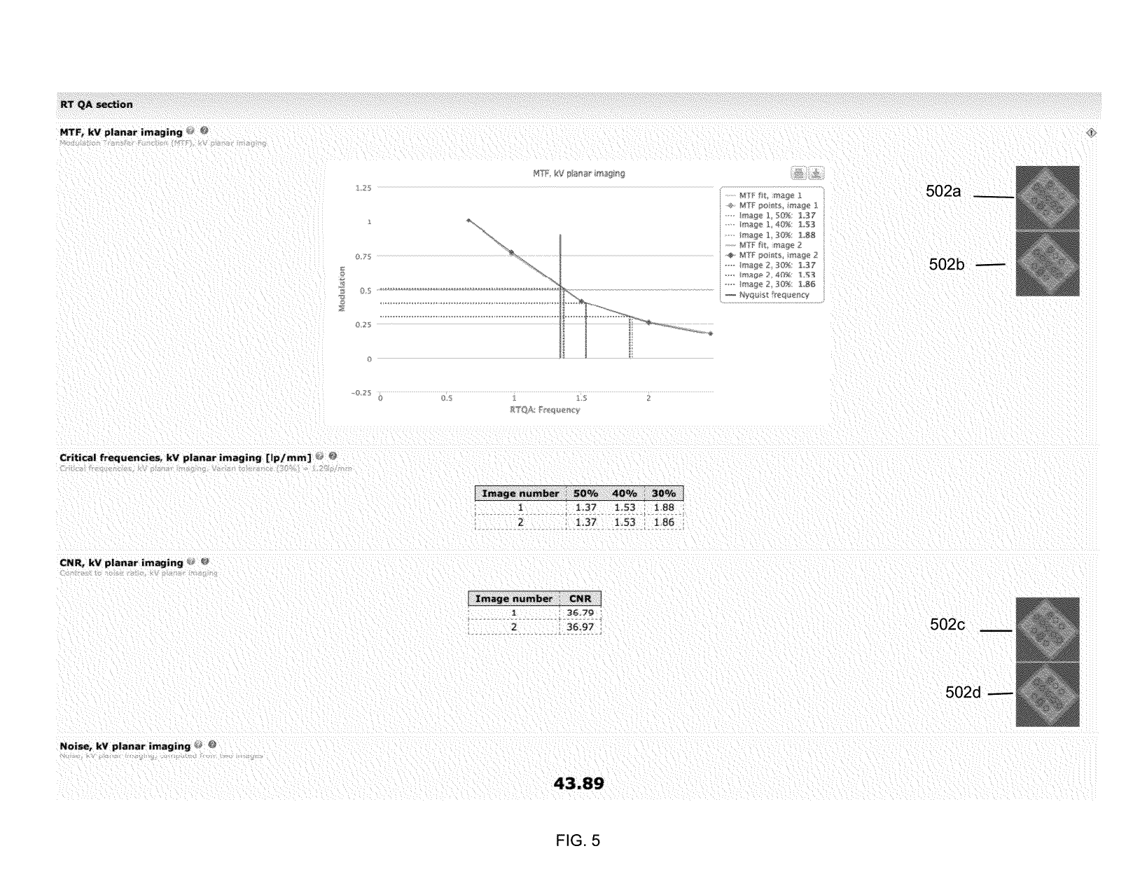

FIG. 5 provides alternative depictions of QA test data, in accordance with aspects described herein. Shown on the right side of the interface are test images (502a-502d) that were captured by the RTT AND IMAGING system, and the corresponding data is presented in a graph. Other representations are possible. In this manner, data can be presented to users in various formats, for instance, numerically, in table(s), bar graphs, temporal graphs, etc., depending on how the user wishes to view the data.

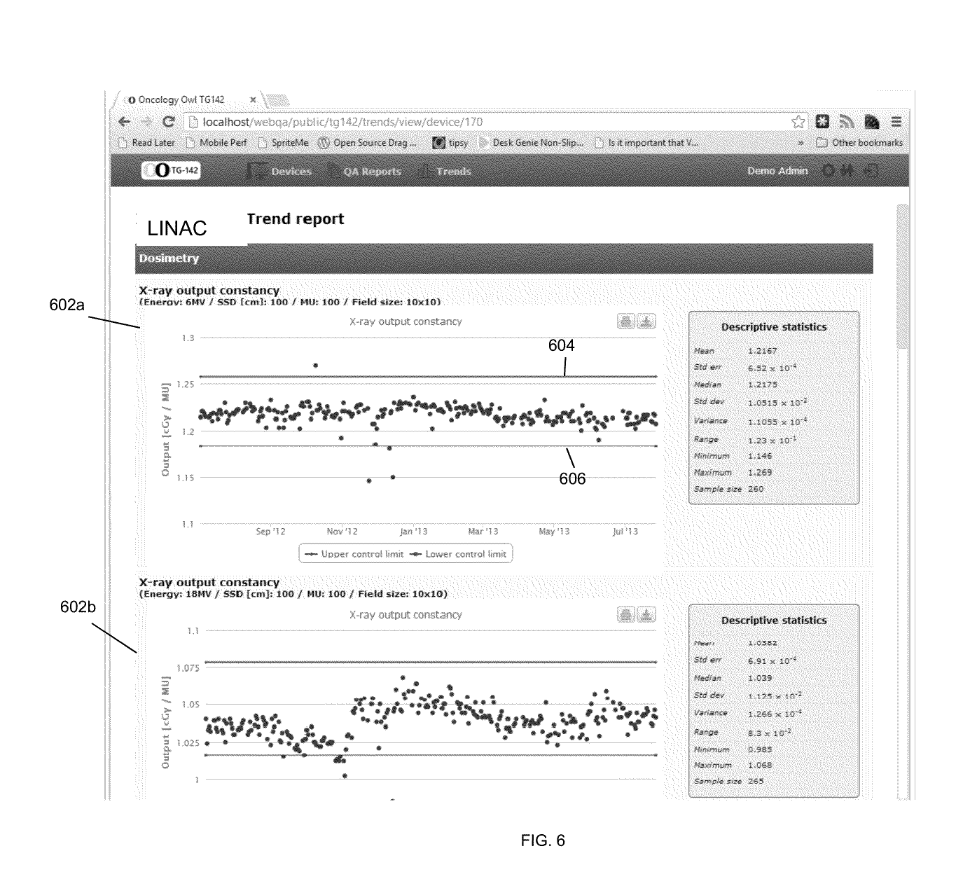

FIG. 6 depicts an example interface displaying a trend report for a particular system (linear accelerator in this example), in accordance with aspects described herein. In the case of a linear accelerator, the radiation being used to treat a patient is an ‘output’ and is measured in centigray (cGy) per unit of time each day. The output levels are tracked over time to ensure that the output is consistent. The graphs 602a and 602b in FIG. 6 show measured output values and upper and lower acceptable limits (+/−2% in this example) for the x-ray output constancy at 6 MV (602a) and 16 MV (602b). The upper and lower acceptable limits for x-ray output constancy at 6 MV are represented by lines 604 and 606, in this example. Each graph shows output as a function of time over the course of several months. Each plotted point represents output for a given day. A user can hover over a point to cause a popup to be displayed indicating the actual output measured for that data point. If the value was out of specification, a note/comment can pop up indicating why the value is out of specification and what was done to rectify the problem.

Data points not within specification (i.e. above the upper limit or below the lower limit) can be highlighted, colored a different color, etc. to draw the user's attention to the data point. In graph 602a for x-ray output constancy at the 6 MV energy level, there are four such data points not within the specification range.

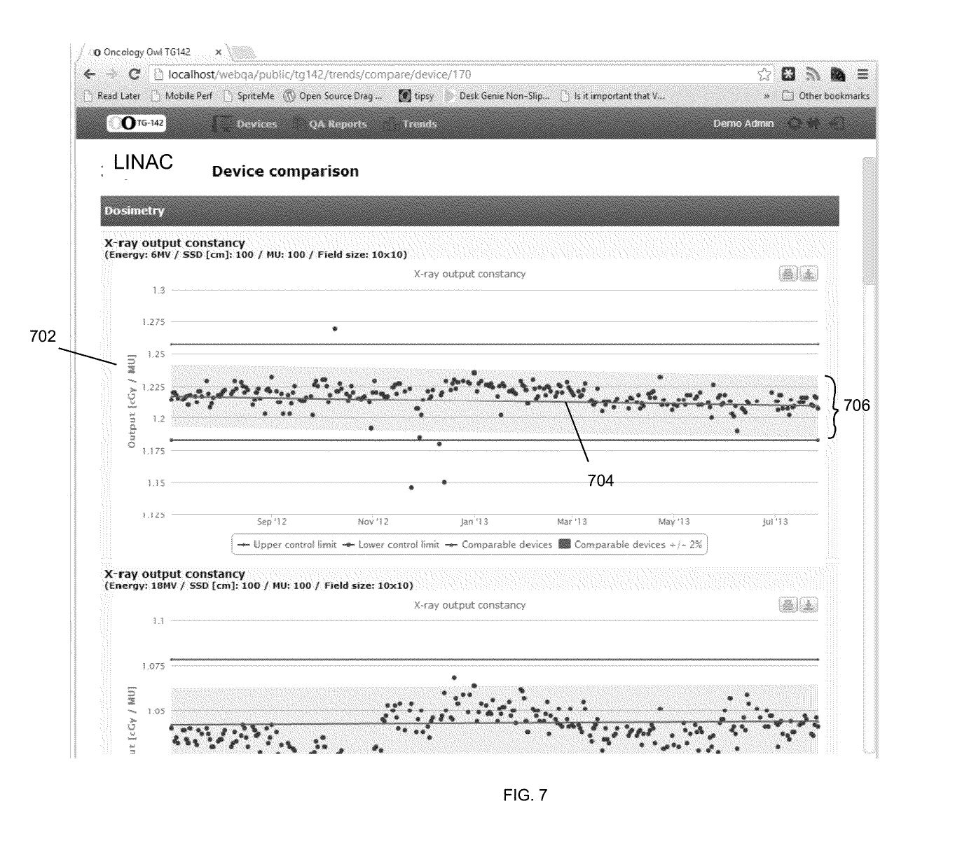

FIG. 7 depicts an example device comparison interface, in accordance with aspects described herein. The device comparison interface displays a comparison of the QA test values (for X-ray output constancy at different MV values in this example) for a particular model machine with the range of QA test values across multiple machines of that model. Since the cloud facility can include QA test data for a plurality of machines of each model, a trend report can be built and presented to show not only how the QA test data for a particular machine is trending over time, but also how it compares to all of the machines (of that model) for which QA test data is stored in the cloud facility. This can be significant because it benchmarks how a particular machine is performing from a QA perspective as compared other machines of that same or similar model. QA data for a particular model machine therefore can serve as a benchmark against which constituent machine's performance can be measured.

In FIG. 7, the plotted data points of comparison graph 702 each indicate a measured x-ray output constancy value for a given test for the machine. A negatively sloped line 704 (of a desired color) indicates historic average output across machines of that model. A shaded region 706 (of a desired color) indicates the range between average upper and lower limits (e.g. within one standard deviation of the average) across machines of that model. In the example of FIG. 7, the subject machine's x-ray output constancy is generally in accordance with x-ray output constancy of the other machines of this model.

In many situations, an RTT AND IMAGING system vendor is not allowed to perform the QA testing and analysis on the RTT AND IMAGING system—it is usually an independent testing group that performs this work, and the person responsible for the testing and analysis is a medical physicist. In the typical scenario, a technician will perform various measurements to test the machine. In the event of a compliance failure or other issue, the physicist will be notified and will address the situation by investigating the error and taking any appropriate corrective action. In one example, this can be aided by a mobile application as described herein.

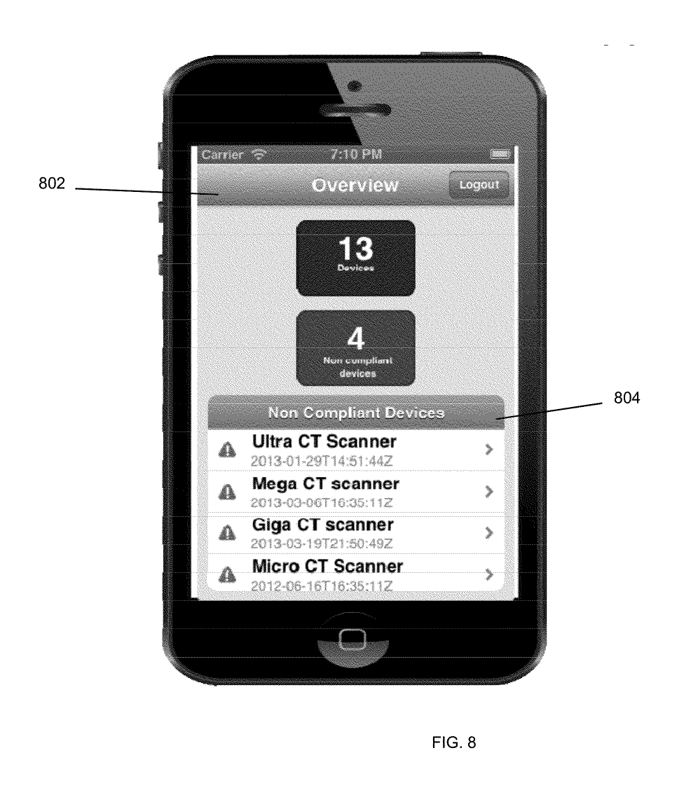

FIG. 8 depicts an example interface of a mobile application, in accordance with aspects described herein. A user logs into the mobile application using account credentials. In one example, the user is a medical physicist responsible for one or more RTT AND IMAGING systems. These RTT AND IMAGING systems are associated with the user's account. In one example, associated with the user account are all RTT AND IMAGING systems associated with a particular entity. For instance, a health care provider may have multiple hospitals, each with one or more RTT AND IMAGING systems. The health care provider may employ a user (medical physicist) responsible for these machines. The user can login to an account and view/manage properties of the RTT AND IMAGING systems.

In the example of FIG. 8, an overview 802 is presented to the user that displays the status of each machine. It shows that 13 devices were tested (in a given timeframe, for instance, such as overnight or early morning). Of those 13 devices, four were found to be out of compliance and these four devices are listed as non-compliant devices 804. The user can select a device listed as a non-compliant device and be presented with an indication of what aspect is out of compliance, and additionally can add notes regarding the compliance failure. For instance, the user can add a note that imaging or treatment using the RTT AND IMAGING system can continue but that a period of time is to be blocked off so that the user can perform diagnostics, such as rechecking the output, servicing the machine by making adjustments, and so forth. Alternatively, the user can issue a note that all use of the equipment is to be cancelled indefinitely. Notifications or other alerts about the issued notes can be automatically provided (through the web-interface, email, text massage, phone, etc.) to the necessary parties, for instance an on-duty technician responsible for operating the equipment in the health care facility.

Subsequently, further notes can be added about any repairs, reconfiguration, retesting, etc. performed on the RTT AND IMAGING system. Additionally, the user can use the application to electronically sign-off on compliance issues that have arisen. All of that activity may be tracked and stored in a cloud facility as described herein. In addition to the above functionality provided by the mobile application, all of the above may be performed through a web interface to the cloud facility. Therefore, any device with a web browser can access the cloud facility and interact with aspects thereof.

As described herein, the cloud facility can handle QA analysis and data management. As part of this, data may be grouped by trends into virtual folders, and methods for process control may be performed on the data. Grouping can be automatic, user-defined, or categorized (such as by machine type), or any combination of the preceding. Further, automated image analysis can be performed on test images obtained by various RTT AND IMAGING systems including CT, CBCT, planar kV/MV, radiation coincidence, and multileaf collimator (MLC) devices, and associated techniques. Images can be recognized automatically and processed with the appropriate algorithm(s).

One example of a set of tools to control a process that can be applied to RTT AND IMAGING systems is Statistical Process Control (SPC). SPC can be used for many purposes, for instance to detect (relatively) large deviations from normal equipment/process operation and provide alerts to technicians or other device operators as to the health or usability of the machines. This is done through the generation of control charts tailored to QA of RTT AND IMAGING systems based on the aggregated data that is collected. Additionally, it can be observed whether data points are trending in a wrong direction, and, if so, this observation can be used as an indicator to predict that something has gone wrong or will go wrong within a predicted time-frame. In this respect, SPC can detect slow drift in machine performance and offer predictive advice directed to the future functioning of the system. The system can notify the responsible party to fix the issue before the equipment goes out of the acceptable standard, to facilitate avoidance of patient mistreatment. Cases of unplanned shut down of the system can therefore be avoided.

In one example, an alert can be immediately issued to an operator informing the operator that a machine having issues (or predicted to have issues in the near future) is not to be used until the issues can be resolved. User-defined rules and action limits (e.g. specifications) can be input to the system by the users in order to setup custom alerts and custom specifications for the systems. Such rules and specifications can be stored in the cloud facility as QA protocols for comparison, automated or otherwise, against test results.

Facilities described herein may be useful for the measurement device (“phantom”) manufacturers through provision of a backend database for these manufacturers to use with their install base (i.e. their customers). Because the QA test data is stored in the cloud facility, these manufacturers can have access to this data. The data can be analyzed to identify opportunities for the measurement device manufacturers to offer their customers new functions, new features, opportunities for upgrade or repair of the measurement devices, and so forth.

Further in this regard, the measurement device manufactures can make use of the aggregated data to identify whether out-of-specification test results are due to an RTT AND IMAGING system failure or whether there may be a problem with a measurement device itself. Opportunities for preventative maintenance are thereby provided. The device manufactures could analyze the aggregated data and assess whether the measurement device is causing the problems. Some measurement devices degrade over time (for instance diodes may have a useful life before they need to be adjusted or replaced). The cloud facility can enable a manufacturer to monitor for device degradation and/or predict when the device starts to degrade. An indication could be sent from the manufacturer to the customer that the device is failing (e.g. the diodes are drifting), to inform the customer to recalibrate or replace the device.

The cloud facility can maintain the QA data and information in a secure/encrypted manner. This includes all measured/calculated RTT AND IMAGING system test data values and uploaded images. It also includes both input and output PDFs, text documents in various formats (such as Microsoft Word format), image files (JPEGs, etc.) and any other files that are uploaded to, or created by, the cloud facility. Additionally, equipment lists and the specifications associated with that equipment, such as user-provided or standardized templates like the TG 142 described above, and account information may be maintained. All of this data can be maintained in a secure manner and in compliance with government regulations and other standards, such as the Health Insurance Portability and Accountability Act (HIPAA).

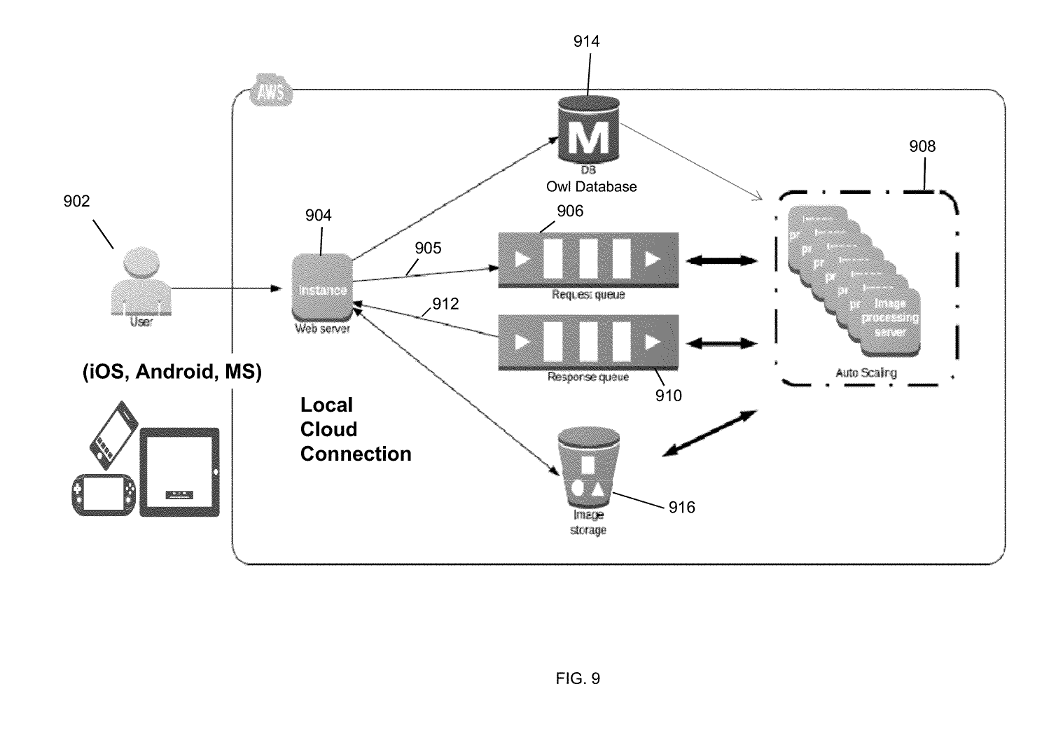

FIG. 9 provides an overview of an example cloud facility in accordance with aspects described herein. The cloud facility may have an architecture that may be certified under various certification schemes, including DICOM, HIPAA, HL7, and CEHRT, as examples. The cloud facility may provide text, email, database, and other network facilities and capabilities.

A user 902 (e.g. a computer system, such as a mobile, desktop, laptop, etc computer system, under operation/control of a user) is in communication with a web server (instance) 904, for instance a webpage through which data may be communicated between the user 902 and components of the cloud facility. The web server 904 places requests 905 on a request queue 906. From the request queue 906, requests 905 are processed by a set of one or more processing servers 908. The set of processing servers 908 may be elastic in that it may auto-scale according to, for instance, the frequency and number of requests coming into the request queue. Rather than queuing data requests and processing them serially, multiple different machines may work on multiple tasks concurrently. The set of processing servers 908 can scale according to the processing load. The processing servers 908 may then return responses to a response queue 910 that provides a response 912 back to the user 902 through the web server 904. Requests 905 may be simple requests to read/write data, or they could be requests for more advanced operations such as analysis of test data or generation of reports.

Additionally, one or more databases (Owl database 914, Image storage database 916) are provided for redundant storage of data, including test data and images acquired using the RTT AND IMAGING systems.

Not pictured are computer system(s) of the RTT AND IMAGING or PACS systems from which test data and images are uploaded to the cloud facility. It is understood, however, that these computer systems may be communicatively coupled to the cloud facility across one or more networks and provide such data through a web server or other intermediary to the cloud facility, such as database(s) and/or request queue(s) thereof.

Cloud technology provides scalable architecture with ease of integration (software, other cloud services), and loosely coupled, elastic, and redundant databases facilitating data resilience. Open source software may be used for coding aspects of the cloud facility software, and various technologies are possible. Below are some example technologies to facilitate aspects of the cloud facility described herein:
PHP 5+: A widely used programming language for web applications; Used for millions of sites (examples: WordPress, Drupal, Joomla; Facebook; Wikipedia); First class cloud citizen (easy to interface with any cloud)—Makes it easy to develop applications quickly
Zend Framework: Extensive application framework facilitating rapid development; Created and maintained by Zend Technologies; Automated testing (Continuous integration); Feature rich
HTML/CSS/Javascript: Industry standards
jQuery: Feature rich Javascript library; Document Object Model (DOM) traversal and manipulation; Handles browser inconsistencies
Highcharts (library of interactive charts)
Amazon Web services: Various services utilized to harness power of the cloud (EC2, RDS, S3, SQS, EBS, Route 53, ELB); Ability to scale to meet demand (elasticity); Loosely coupled

Use of a cloud architecture advantageously enables, as examples:
Agility—Easy access to customers; Easy collaborations; Instant service and upgrades
Scalability—Scales to company growth and seasonal need
Elasticity—On demand upload, processing, and storage services
Redundancy—data shared over multiple centers world wide
Encrypted data
Regulatory support—Evolving HIPAA requirements can be met and insurance provided

Aspects of the present invention may be embodied in one or more systems, one or more methods and/or one or more computer program products. In some embodiments, aspects of the present invention may be embodied entirely in hardware, entirely in software (for instance in firmware, resident software, micro-code, etc.), or in a combination of software and hardware aspects that may all generally be referred to herein as a “system” and include circuit(s) and/or module(s).

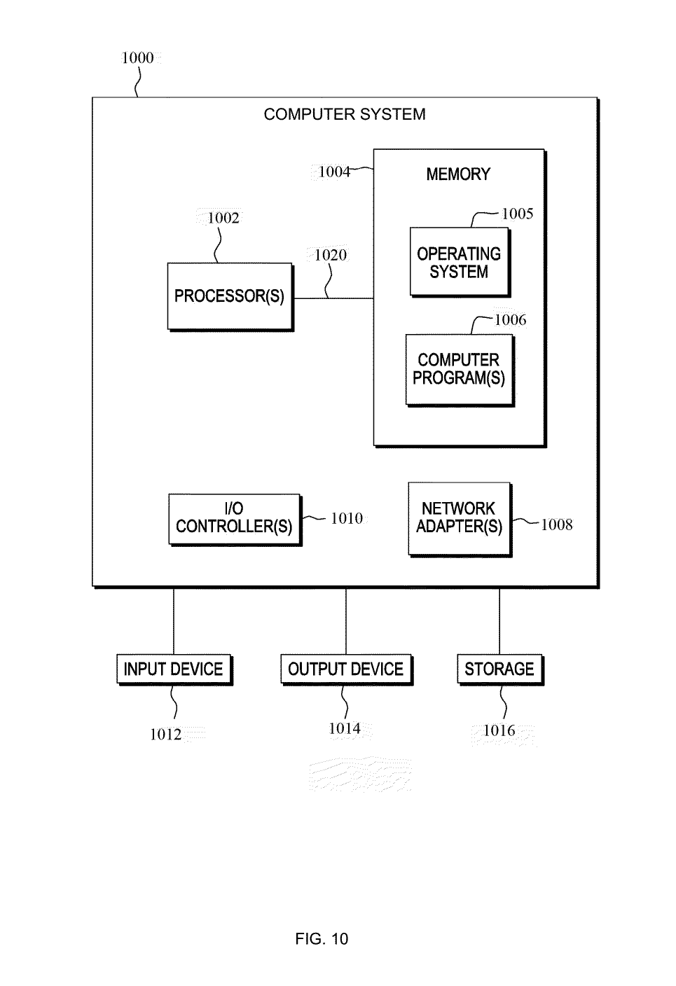

FIG. 10 depicts one example of a computer system to incorporate and use aspects described herein. Computer system 1000 is suitable for storing and/or executing program code, such as program code for performing processes described herein, and includes at least one processor 1002 coupled directly or indirectly to memory 1004 through, a bus 1020. In operation, processor(s) 1002 obtain from memory 1004 one or more instructions for execution by the processors. Memory 1004 may include local memory employed during actual execution of the program code, bulk storage, and cache memories which provide temporary storage of at least some program code in order to reduce the number of times code must be retrieved from bulk storage during program code execution. A non-limiting list of examples of memory 1004 includes a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. Memory 1004 includes an operating system 1005 and one or more computer programs 1006 for performing aspects described herein.

Input/Output (I/O) devices 1012, 1014 (including but not limited to keyboards, displays, pointing devices, etc.) may be coupled to the system either directly or through I/O controllers 1010.

Network adapters 1008 may also be coupled to the system to enable the computer system to become coupled to other computer systems through intervening private or public networks. Modems, cable modem and Ethernet cards are just a few of the currently available types of network adapters 1008. In one example, network adapters 1008 facilitate communicating data between the computer systems, such as data flowing from/to a cloud facility.

Computer system 1000 may be coupled to storage 1016 (e.g., a non-volatile storage area, such as magnetic disk drives, optical disk drives, a tape drive, etc.), having one or more databases. Storage 1016 may include an internal storage device or an attached or network accessible storage. Computer programs in storage 1016 may be loaded into memory 1004 and executed by a processor 1002.

The computer system 1000 may include fewer components than illustrated, additional components not illustrated herein, or some combination of the components illustrated and additional components. Computer system 1000 may include any computing device known in the art, such as a mainframe, server, personal computer, workstation, laptop, handheld computer, mobile computer, smartphone, telephony device, network appliance, virtualization device, storage controller, etc.

In addition, processes described above may be performed by multiple computer systems 1000, working as part of a clustered computing environment.

In some embodiments, aspects described herein may take the form of a computer program product embodied in one or more computer readable medium(s). The one or more computer readable medium(s) may have embodied thereon computer readable program code. Various computer readable medium(s) or combinations thereof may be utilized. For instance, the computer readable medium(s) may comprise a computer readable storage medium, examples of which include (but are not limited to) one or more electronic, magnetic, optical, or semiconductor systems, apparatuses, or devices, or any suitable combination of the foregoing. Example computer readable storage medium(s) include, for instance: an electrical connection having one or more wires, a portable computer diskette, a hard disk or mass-storage device, a random access memory (RAM), read-only memory (ROM), and/or erasable-programmable read-only memory such as EPROM or Flash memory, an optical fiber, a portable compact disc read-only memory (CD-ROM), an optical storage device, a magnetic storage device (including a tape device), or any suitable combination of the above. A computer readable storage medium is defined to comprise a tangible medium that can contain or store program code for use by or in connection with an instruction execution system, apparatus, or device, such as a processor. The program code stored in/on the computer readable medium therefore produces an article of manufacture (such as a “computer program product”) including program code.

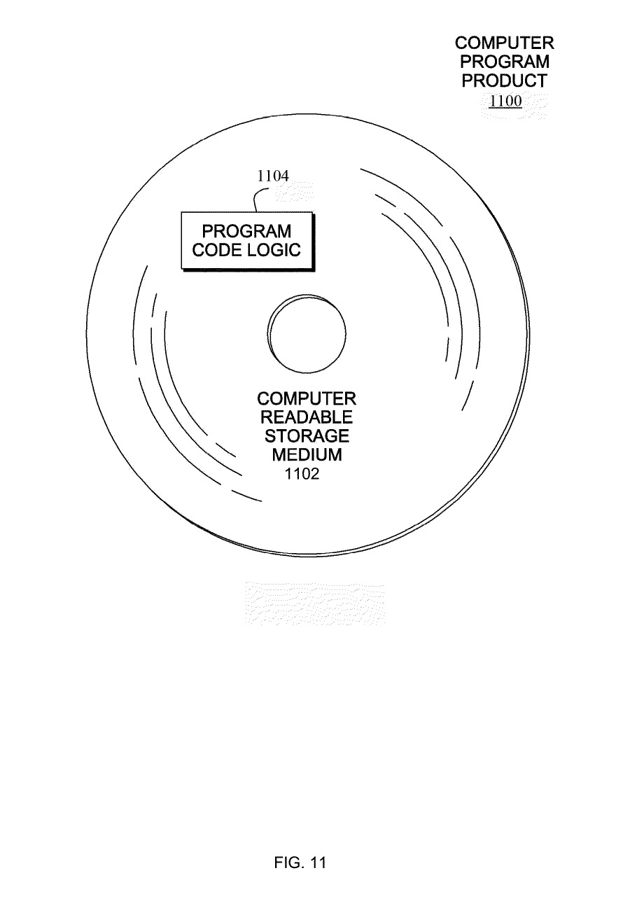

Referring now to FIG. 11, in one example, a computer program product 1100 includes, for instance, one or more computer readable media 1102 to store computer readable program code means or logic 1104 thereon to provide and facilitate one or more aspects of the present invention.

Program code contained or stored in/on a computer readable medium can be obtained and executed by a computer system (computer, computer system, etc. including a component thereof) and/or other devices to cause the computer system, component thereof, and/or other device to behave/function in a particular manner. The program code can be transmitted using any appropriate medium, including (but not limited to) wireless, wireline, optical fiber, and/or radio-frequency. Program code for carrying out operations to perform, achieve, or facilitate aspects of the present invention may be written in one or more programming languages. In some embodiments, the programming language(s) include object-oriented and/or procedural programming languages such as C, C++, C#, Java, etc. Program code may execute entirely on the user's computer, entirely remote from the user's computer, or a combination of partly on the user's computer and partly on a remote computer. In some embodiments, a user's computer and a remote computer are in communication via a network such as a local area network (LAN) or a wide area network (WAN), and/or via an external computer (for example, through the Internet using an Internet Service Provider).

In one example, program code includes one or more program instructions obtained for execution by one or more processors. Computer program instructions may be provided to one or more processors of, e.g., one or more computer system, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

The flowcharts and block diagrams depicted and described with reference to the Figures illustrate the architecture, functionality, and operation of possible embodiments of systems, methods and/or computer program products according to aspects of the present invention. These flowchart illustrations and/or block diagrams could, therefore, be of methods, apparatuses (systems), and/or computer program products according to aspects of the present invention.

In some embodiments, as noted above, each block in a flowchart or block diagram may represent a module, segment, or portion of code, which comprises one or more executable instructions for implementing the specified behaviors and/or logical functions of the block. Those having ordinary skill in the art will appreciate that behaviors/functions specified or performed by a block may occur in a different order than depicted and/or described, or may occur simultaneous to, or partially/wholly concurrent with, one or more other blocks. Two blocks shown in succession may, in fact, be executed substantially concurrently, or the blocks may sometimes be executed in the reverse order. Additionally, each block of the block diagrams and/or flowchart illustrations, and combinations of blocks in the block diagrams and/or flowchart illustrations, can be implemented wholly by special-purpose hardware-based systems, or in combination with computer instructions, that perform the behaviors/functions specified by a block or entire block diagram or flowchart.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has” and “having”), “include” (and any form of include, such as “includes” and “including”), and “contain” (and any form contain, such as “contains” and “containing”) are open-ended linking verbs. As a result, a method or device that “comprises”, “has”, “includes” or “contains” one or more steps or elements possesses those one or more steps or elements, but is not limited to possessing only those one or more steps or elements. Likewise, a step of a method or an element of a device that “comprises”, “has”, “includes” or “contains” one or more features possesses those one or more features, but is not limited to possessing only those one or more features. Furthermore, a device or structure that is configured in a certain way is configured in at least that way, but may also be configured in ways that are not listed.

The description of the present invention has been presented for purposes of illustration and description, but is not intended to be exhaustive or limited to aspects of the invention in the form disclosed. Many modifications and variations will be apparent to those of ordinary skill in the art without departing from the scope and spirit of aspects of the invention. The embodiment was chosen and described in order to best explain aspects of the principles of the invention and the practical application, and to enable others of ordinary skill in the art to understand aspects of the invention for various embodiments with various modifications as are suited to the particular use contemplated.

What is claimed is:

1. A method comprising:
obtaining test data, the test data comprising results related to radiologic or diagnostic imaging performance of radiation therapy or diagnostic imaging equipment;
performing analysis of the test data against maintained quality assurance (QA) specifications to obtain QA results for the equipment; and
presenting the obtained QA results to a user.

2. The method of claim 1, wherein the presenting comprises:
providing an alert to the user, the alert indicating one or more QA results, of the obtained QA results, that are out-of-specification; or
providing a trend report to the user, the trend report providing a comparison of the obtained QA results to prior-obtained QA results.

3. The method of claim 2, wherein the presenting presents the alert to the user, and wherein the one or more QA results indicated by the alert identify a particular data point or test data value corresponding to that data point that is out of specification.

4. The method of claim 2, wherein the method further comprises aggregating the obtained QA results with additional QA results for other radiation therapy or diagnostic imaging equipment.

5. The method of claim 4, wherein prior-obtained QA results comprises the additional QA results for the other radiation therapy or diagnostic imaging equipment, and wherein the presenting presents the trend report to the user, and wherein the trend report provides a comparison of the obtained QA results to the additional QA results.

6. The method of claim 5, wherein the obtained QA results are for one system and the additional QA results are for one or more other systems, the one system and the one or more other systems being of a same type of equipment, wherein the trend report indicates how QA results for the one system are trending over time, and the trend report indicates how QA results for the one system compare to QA results for the one or more other systems.

7. The method of claim 2, further comprising, in response to the presenting, receiving one or more comments from the user.

8. The method of claim 7, wherein the one or more comments are associated with a QA result, of the obtained QA results, or with an individual test data point, wherein the one or more comments support, explain, resolve, comment-on, or sign-off on the QA result or individual test data point.

9. The method of claim 7, wherein the one or more comments comprises an indication of a repair, reconfiguration, or retest performed on the equipment based on the presenting.

10. The method of claim 7, wherein the one or more comments comprise an electronic signing-off by the user for a compliance issue indicated by QA results.

11. The method of claim 7, wherein the presenting presents a message or description of a compliance issue indicated by the QA results, wherein the one or more comments comprise a comment directed to that compliance issue.

12. The method of claim 1, wherein the analysis comprises statistical process control (SPC) analysis, the SPC analysis facilitating detection of slow drift in machine performance, and wherein the QA results comprises an indication of the slow drift or predictive advice directed to further functioning of the equipment.

13. The method of claim 1, wherein the maintained QA specifications comprise a protocol for QA testing of the equipment, and wherein the method further comprises:
determining whether the obtained test data for the equipment satisfies the protocol; and
providing an indication to the user about any deviation from the protocol.

14. The method of claim 1, further comprising maintaining a Wiki with information about aspects of a facility in which the equipment is maintained, specification about the equipment components, information about test data points of QA testing to be performed for the equipment, or information explaining results of testing performed for the equipment.

15. A computer system comprising:
a memory; and
a processor in communication with the memory, wherein the computer system is configured to perform:
obtaining test data, the test data comprising results related to radiologic or diagnostic imaging performance of radiation therapy or diagnostic imaging equipment;
performing analysis of the test data against maintained quality assurance (QA) specifications to obtain QA results for the equipment; and
presenting the obtained QA results to a user.

16. A computer program product to facilitate secure execution of an executable comprising an encrypted instruction, the computer program product comprising:
a non-transitory computer-readable storage medium comprising program instructions for execution by a processor to perform:
obtaining test data, the test data comprising results related to radiologic or diagnostic imaging performance of radiation therapy or diagnostic imaging equipment;
performing analysis of the test data against maintained quality assurance (QA) specifications to obtain QA results for the equipment; and
presenting the obtained QA results to a user.
